(19) United States
(12) Patent Application Publication
(10) Pub. No.: US 2025/0177410 A1
(43) Pub. Date: Jun. 5, 2025

(54) COMBINATION THERAPIES COMPRISING A SOS1 INHIBITOR AND AN EGFR INHIBITOR

(71) Applicant: Mirati Therapeutics, Inc., San Diego, CA (US)

(72) Inventors: Jacob Haling, San Diego, CA (US); Shilpi Khare, San Diego, CA (US)

(21) Appl. No.: 18/851,642

(22) PCT Filed: Mar. 13, 2023
(86) PCT No.: PCT/US2023/015097

§ 371 Date: Sep. 26, 2024

Related U.S. Application Data
(60) Provisional application No. 63/329,090, filed on Apr. 8, 2022.

(51) Int. Cl. A61K 31/5377; A61K 31/506; A61P 35/00

(52) U.S. Cl. CPC A61K 31/5377; A61K 31/506; A61P 35/00

(57) ABSTRACT

The present invention relates to combination therapies for treating cancers associated with genetic alterations of the MAPK pathway and/or EGFR In particular, the present invention relates to methods of treating cancer in a subject in need thereof, comprising administering to the subject a therapeutically effective amount of a combination of a SOS1 inhibitor and an EGFR inhibitor, pharmaceutical compositions comprising a such compositions, kits comprising such compositions and methods of use therefor.

### FIELD OF THE INVENTION

The present invention relates to combination therapies useful for treating cancer. In particular, the present invention relates to therapeutically effective combinations of a Son of sevenless homolog 1 (SOS1) inhibitor and an EGFR inhibitor, pharmaceutical compositions comprising the inhibitors, kits comprising the compositions and methods of use therefor.

### BACKGROUND OF THE INVENTION

### SOS1 Inhibitors

The Ras family comprises v-Ki-ras2 Kirsten rat sarcoma viral oncogene homolog (KRas), neuroblastoma RAS viral oncogene homolog (NRAS), and Harvey murine sarcoma virus oncogene (HRas) and critically regulates cellular division, growth and function in normal and altered states including cancer (see e.g., Simanshu et al. Cell, 2017. 170 (1): p. 17-33; Matikas et al., Crit Rev Oncol Hematol, 2017. 110: p. 1-12). RAS proteins are activated by upstream signals, including receptor tyrosine kinases (RTKs), and transduce signals to several downstream signaling pathways such as the mitogen-activated protein kinase (MAPK)/extracellular signal-regulated kinases (ERK) pathway. Hyperactivation of RAS signaling is frequently observed in cancer as a result of mutations or alterations in RAS genes or other genes in the RAS pathway. The identification of strategies to inhibit RAS and RAS signaling are predicted to be useful for the treatment of cancer and RAS-regulated disease states.

RAS proteins are guanosine triphosphatases (GTPases) that cycle between an inactive, guanosine diphosphate (GDP)-bound state and an active guanosine triphosphate (GTP)-bound state. RAS proteins exhibit both intrinsic GTP hydrolysis and nucleotide exchange, which is further enhanced by extrinsic GTPase activating proteins (GAPs) and guanine echange factors (GEFs). Son of sevenless homolog 1 (SOS1) is a GEF that mediates the exchange of GDP for GTP, thereby activating RAS proteins. This regulation through GAPs and GEFs is the mechanism whereby activation and deactivation are tightly regulated under normal conditions. Mutations at several residues in all three RAS proteins are frequently observed in cancer and result in RAS remaining predominantly in the activated state (Sanchez-Vega et al., Cell, 2018. 173: p. 321-337 Li et al., Nature Reviews Cancer, 2018. 18: p. 767-777). Mutations at codon 12 and 13 disrupt the GTP hydrolysis and exchange rate of RAS proteinse t. Recent biochemical analysesdemonstrated these mutated proteins still require nucleotide cycling for activation based on their intrinsic GTPase activity and may exhibit partial sensitivity to extrinsic GAPs and GEFs. As such, mutant RAS proteins are sensitive to inhibition of upstream factors such as the SOS1 GEF (Hillig, 2019; Patricelli, 2016; Lito, 2016; Nichols, 2018).

The three main RAS-GEF families that have been identified in mammalian cells are SOS, RAS-GRF and RAS-GRP (Rojas, 2011). RAS-GRF and RAS-GRP are expressed in the cells of the central nervous system and hematopoietic cells, respectively, while the SOS family is ubiquitously expressed and is responsible for transducing RTK signaling. The SOS family comprises SOS1 and SOS2 and these proteins share approximately 70% sequence identity. SOS1 appears to be much more active than SOS2 due to the rapid degradation of SOS2. The mouse SOS2 knockout is viable whereas the SOS1 knockout is embryonic lethal. A tamoxifen-inducible SOS1 knockout mouse model was used to interrogate the role of SOS1 and SOS2 in adult mice and demonstrated the SOS1 knockout was viable but the SOS1/2 double knockout was not viable (Baltanas, 2013) suggesting functional redundancy and that selective inhibition of SOS1 may have a sufficient therapeutic index for the treatment of SOS1-RAS activated diseases.

SOS proteins are recruited to phosphorylated RTKs through an interaction with growth factor receptor bound protein 2 (GRB2). Recruitment to the plasma membrane places SOS in close proximity to RAS and enables SOS-mediated RAS activation. SOS proteins bind to RAS through a catalytic binding site that promotes nucleotide exchange as well as through an allosteric site that binds GTP-bound RAS-family proteins which increases the catalytic function of SOS (Freedman et al., Proc. Natl. Acad. Sci, USA 2006. 103 (45): p. 16692-97). Binding to the allosteric site relieves steric occlusion of the catalytic site and is therefore required for full activation of the catalytic site. Retention of the active conformation at the catalytic site following interaction with the allosteric site is maintained in isolation due to strengthened interactions of key domains in the activated state. SOS1 mutations are found in Noonan syndrome and several cancers including lung adenocarcinoma, embryonal rhabdomyosarcoma, Sertoli cell testis tumor and granular cell tumors of the skin (see e.g., Denayer, E., et al, Genes Chromosomes Cancer, 2010. 49 (3): p. 242-52).

GTPase-activating proteins (GAPs) are proteins that stimulate the low intrinsic GTPase activity of RAS family members and therefore converts active GTP-bound RAS proteins into inactive, GDP-bound RAS proteins (e.g., see Simanshu, D. K., Cell, 2017, Ras Proteins and their Regulators in Human Disease). While activating alterations in the phosphatase PTPN11 (SHP2) and the GEF SOS1 occur in cancers, inactivating mutations and loss-of-function alterations in the GAP neurofibromin 1 (NF-1) also occur creating a state where SOS1 activity is unopposed and activity downstream of the pathway through RAS proteins is elevated.

### EGFR Inhibitors

Epidermal Growth Factor Receptor (EGFR) is a transmembrane protein tyrosine kinase of the ErbB receptor family. Upon binding epidermal growth factor (EGF), the EGFR receptor can homo-dimerize with another EGFR molecule or hetero-dimerize with another family member such as ErbB2 (HER2), ErbB3 (HER3), or ErbB4 (HER4). Homo- and/or hetero-dimerization of ErbB receptors results in the phosphorylation of key tyrosine residues in the intracellular domain and leads to the stimulation of numerous intracellular signal transduction pathways involved in cell proliferation and survival.

Overexpression of the EGFR gene has been identified in a variety of cancers including bladder, brain, head and neck, pancreas, lung, breast, ovary, colon, prostate, and kidney. In addition to overexpression, EGFR activating mutations have been detected in a subset of non-small cell lung cancers (NSCLCs) tumors. These mutations tend to occur within EGFR exons 18-21, which encodes a portion of the EGFR kinase domain. Approximately 90% of these mutations are exon 19 deletions or exon 21 L858R point mutations (Ladanyi and Pao (2008) Mod Path. May; 21 Suppl 2: S16-22. doi: 10.1038/modpathol.3801018). These mutations increase the kinase activity of EGFR, leading to hyperactivation of downstream pro-survival signaling pathways.

The frequency of overexpression and/or activating mutations of EGFR has made it a desired target for anticancer therapies and a number of EGFR inhibitors have been developed and are clinically available.

First generation erlotinib and gefitinib inhibit EGFR activity by competitively binding to the ATP binding site of the EGFR kinase domain; however additional mutations in the EGFR gene, e.g., the T790M mutation, produces mutant EGFR proteins to which drugs like erlotinib and gefitinib bind less well. Those mutations are associated with resistance to the drugs and to relapse in cancer patients bearing such mutation leading to the development of second generation EGFR inhibitors targeting the T790M mutant.

Osimertinib is an irreversible, third-generation epidermal growth factor receptor (EGFR) inhibitor that is highly selective for EGFR-activating mutations as well as the EGFR T790M gate-keeper mutation in patients with advanced non-small cell lung cancer (NSCLC) with EGFR oncogene addiction. Despite the documented efficacy of osimertinib in a clinical setting, patients inevitably develop resistance. Our data suggests that the combination of MRTX0902 with EGFR inhibitors such as osimertinib may delay the onset of acquired resistance. Here we demonstrate that the SOS1 inhibitor MRTX0902 can increase the depth of response to osimertinib in an EGFR addicted NSCLC xenograft model.

Furthermore, inhibition of the pathway-related enzyme MEK results in increased expression of ErbB family members, especially EGFR, that can lead to adaptive and acquired resistance to ErbB family inhibitors (Sun et al., (2014) Cell Reports 7:86-93).

### SUMMARY OF THE INVENTION

The combination therapy of the present invention, in one aspect, synergistically increases the potency of SOS1 inhibitors resulting in improved efficacy of SOS1 inhibitors disclosed herein. The combination therapy of the present invention, in another aspect, provides improved clinical benefit to patients compared to treatment with SOS1 inhibitors disclosed herein as a single agent.

Thus, in one aspect of the invention there are provided therapeutically effective combinations of a SOS1 inhibitor such as those described in WO2021/127429, WO2021/173524, WO2022/026465, U.S. provisional patent application 63/213,112 (and corresponding national and international applications and publications) and as described in greater detail herein, for instance:

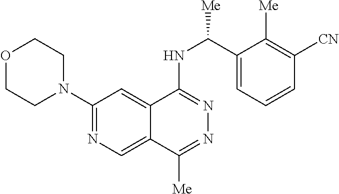 [SMILES: Cc1c(C#N)cccc1[C@@H](C)Nc1nnc(C)c2cnc(N3CCOCC3)cc12]

(R)-2-methyl-3-(1-((4-methyl-7-morpholinopyrido[3,4-d]pyridazin-1-yl)amino)ethyl)benzonitrile,

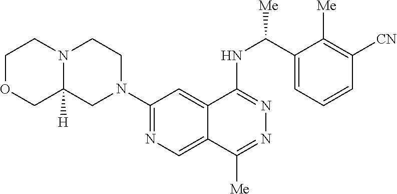 [SMILES: Cc1c(C#N)cccc1[C@@H](C)Nc1nnc(C)c2cnc(N3CCN4CCOC[C@@H]4C3)cc12]

3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile,

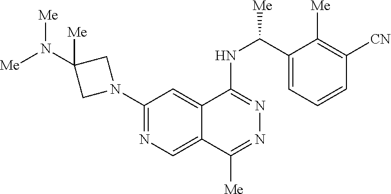 [SMILES: Cc1c(C#N)cccc1[C@@H](C)Nc1nnc(C)c2cnc(N3CC(C)(N(C)C)C3)cc12]

(R)-3-(1-((7-(3-(dimethylamino)-3-methylazetidin-1-yl)-4-methylpyrido[3,4-d]pyridazin-1-yl)amino)ethyl)-2-methylbenzonitrile,

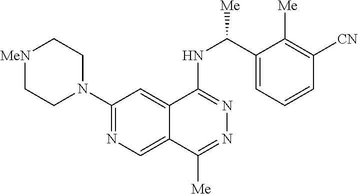 [SMILES: Cc1c(C#N)cccc1[C@@H](C)Nc1nnc(C)c2cnc(N3CCN(C)CC3)cc12]

(R)-2-methyl-3-(1-((4-methyl-7-(4-methylpiperazin-1-yl)pyrido[3,4-d]pyridazin-1-yl)amino)ethyl)benzonitrile,

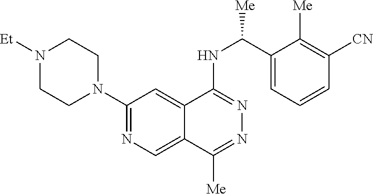 [SMILES: CCN1CCN(c2cc3c(N[C@H](C)c4cccc(C#N)c4C)nnc(C)c3cn2)CC1]

(R)-3-(1-((7-(4-ethylpiperazin-1-yl)-4-methylpyrido[3,4-d]pyridazin-1-yl)amino)ethyl)-2-methylbenzonitrile,

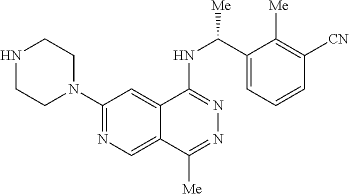 [SMILES: Cc1c(C#N)cccc1[C@@H](C)Nc1nnc(C)c2cnc(N3CCNCC3)cc12]

(R)-2-methyl-3-(1-((4-methyl-7-(piperazin-1-yl)pyrido[3,4-d]pyridazin-1-yl)amino)ethyl)benzonitrile,

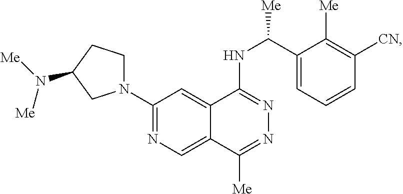 [SMILES: Cc1c(C#N)cccc1[C@@H](C)Nc1nnc(C)c2cnc(N3CC[C@H](N(C)C)C3)cc12]

3-((R)-1-((7-((S)-3-(dimethylamino) pyrrolidin-1-yl)-4-methylpyrido[3,4-d]pyridazin-1-yl)amino)ethyl)-2-methylbenzonitrile,

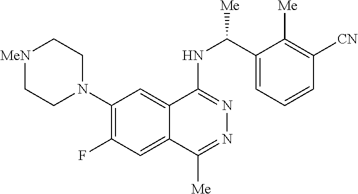 [SMILES: Cc1c(C#N)cccc1[C@@H](C)Nc1nnc(C)c2cc(F)c(N3CCN(C)CC3)cc12]

(R)-3-(1-((6-fluoro-4-methyl-7-(4-methylpiperazin-1-yl) phthalazin-1-yl)amino)ethyl)-2-methylbenzonitrile,
   or a pharmaceutically acceptable salt thereof, and an EGFR inhibitor compound such as osimertinib (TAGRISSO®), gefitinib, erlotinib, afatinib, brigatinib, icotinib, cetuximab and other small and large molecule EGFR inhibitors, or a pharmaceutically acceptable salt thereof.

In another aspect of the invention there are provided therapeutically effective combinations of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and an EGFR inhibitor selected from osimertinib (TAGRISSO®), gefitinib, erlotinib, afatinib, brigatinib, icotinib, cetuximab and other small and large molecule EGFR inhibitors, or a pharmaceutically acceptable salt thereof.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the EGFR inhibitor compound osimertinib, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the EGFR inhibitor compound gefitinib, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the EGFR inhibitor compound erlotinib, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the EGFR inhibitor compound afatinib, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the EGFR inhibitor compound brigatinib, or a pharmaceutically acceptable salt thereof, and a pharmaceutically acceptable excipient.

In another aspect of the invention, pharmaceutical compositions are provided for use in the methods comprising a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile or a pharmaceutically acceptable salt thereof, and the EGFR inhibitor compound icotinib, or a pharmaceutically acceptable salt thereof, and a pharmaceutically acceptable excipient.

In one aspect of the invention, provided herein are methods of treating cancer in a subject in need thereof, comprising administering to the subject a therapeutically effective amount of a combination of a SOS1 inhibitor such as (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino) pyrrolidin-1-yl)-4-methylpyrido[3,4-d]pyridazin-1-yl)amino)ethyl)-2-methylbenzonitrile or (R)-3-(1-((6-fluoro-4-methyl-7-(4-methylpiperazin-1-yl) phthalazin-1-yl)amino)ethyl)-2-methylbenzonitrile. and the EGFR inhibitor selected from osimertinib (TAGRISSO®), gefitinib, erlotinib, afatinib, brigatinib, icotinib, cetuximab and other small and large molecule EGFR inhibitors, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the EGFR inhibitor osimertinib, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the EGFR inhibitor gefitinib, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the EGFR inhibitor erlotinib, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the EGFR inhibitor afatinib, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the EGFR inhibitor brigatinib, or a pharmaceutically acceptable salt or a pharmaceutical composition thereof.

In one aspect of the invention, provided herein are methods of treating cancer in a subject in need thereof, comprising administering to the subject a therapeutically effective amount of a combination of the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the EGFR inhibitor icotinib, or a pharmaceutically acceptable salt or a pharmaceutical composition thereof.

In one embodiment, the cancer is a SOS1-associated cancer.

In one embodiment, the cancer is a KRas G12C-associated cancer.

In one embodiment, the cancer is selected from the group consisting of lung cancer, leukemia, colorectal cancer, uterine cancer and pancreatic cancer . . .

In one embodiment, the cancer is selected from the group consisting of lung cancer and leukemia.

In one embodiment, the lung cancer is lung adenocarcinoma.

In one embodiment, lung cancer is non-small cell lung cancer.

In one embodiment, leukemia is acute myeloid leukemia (AML).

In one embodiment, the SOS1-associated cancer is lung cancer.

In one embodiment, the pancreatic cancer is ductal carcinoma of the pancreas.

In some aspects of the invention, EGFR inhibitor(s) and SOS1 inhibitor(s) are the only active agents in the provided compositions and methods.

Examples of SOS1 inhibitors suitable for the provided compositions and methods include, but are not limited to (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino) pyrrolidin-1-yl)-4-methylpyrido[3,4-d]pyridazin-1-yl)amino)ethyl)-2-methylbenzonitrile and (R)-3-(1-((6-fluoro-4-methyl-7-(4-methylpiperazin-1-yl) phthalazin-1-yl)amino)ethyl)-2-methylbenzonitrile, and pharmaceutically acceptable salts thereof. In yet another aspect, the invention provides for methods for increasing the sensitivity of a cancer cell to a SOS1 inhibitor, or to an EGFR inhibitor, comprising contacting the cancer cell with a therapeutically effective amount of a combination of an EGFR inhibitor compound such as osimertinib (TAGRISSO®), gefitinib, erlotinib, afatinib, brigatinib, icotinib, cetuximab and other small and large molecule EGFR inhibitors, or a pharmaceutically acceptable salt or a pharmaceutical composition thereof, and a SOS1 inhibitor or a pharmaceutically acceptable salt or a pharmaceutical composition thereof, including the SOS1 inhibitors described herein such as (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino) pyrrolidin-1-yl)-4-methylpyrido[3,4-d]pyridazin-1-yl)amino)ethyl)-2-methylbenzonitrile and (R)-3-(1-((6-fluoro-4-methyl-7-(4-methylpiperazin-1-yl) phthalazin-1-yl)amino)ethyl)-2-methylbenzonitrile, and pharmaceutically acceptable salts thereof, to synergistically increase the sensitivity of the cancer cell to the SOS1 or EGFR inhibitor. In one embodiment, the contacting is in vitro. In one embodiment, the contacting is in vivo.

Also provided herein are methods for treating cancer in a subject in need thereof, the method comprising (a) determining that cancer is associated with a SOS1 mediated cancer and/or genetic alterations of the MAPK pathway (e.g., a SOS1-associated cancer) (e.g., as determined using a regulatory agency-approved, e.g., FDA-approved, assay or kit); and (b) administering to the patient a therapeutically effective amount of a combination of a SOS1 inhibitor, or a pharmaceutically acceptable salt or a pharmaceutical composition thereof, and an EGFR inhibitor or a pharmaceutically acceptable salt or a pharmaceutical composition thereof, wherein the EGFR inhibitor synergistically increases the sensitivity of the SOS1-associated cancer to the SOS1 inhibitor, for instance to (R)-2-methyl-3-(1-((4-methyl-7-morpholinopyrido[3,4-d]pyridazin-1-yl)amino)ethyl)benzonitrile.

Also provided herein are kits comprising a SOS1 inhibitor, or a pharmaceutically acceptable salt or a pharmaceutical composition thereof and an EGFR inhibitor compound or a pharmaceutically acceptable salt or a pharmaceutical composition thereof. Also provided is a kit comprising the SOS1 inhibitor (R)-2-methyl-3-(1-((4-methyl-7-morpholinopyrido[3,4-d]pyridazin-1-yl)amino)ethyl)benzonitrile, or a pharmaceutically acceptable salt or a pharmaceutical composition thereof, and the EGFR inhibitor compound osimertinib or a pharmaceutically acceptable salt or a pharmaceutical composition thereof, for use in treating a SOS1-associated cancer.

In a related aspect, the invention provides a kit containing a dose of a SOS1 inhibitor, or a pharmaceutically acceptable salt or a pharmaceutical composition thereof and an EGFR inhibitor compound or a pharmaceutically acceptable salt or a pharmaceutical composition thereof in an amount effective to inhibit proliferation of cancer cells in a subject. The kit in some cases includes an insert with instructions for administration of a SOS1 inhibitor, or a pharmaceutically acceptable salt or a pharmaceutical composition thereof and an EGFR inhibitor compound or a pharmaceutically acceptable salt or a pharmaceutical composition thereof. The insert may provide a user with one set of instructions for using the a SOS1 inhibitor or a pharmaceutically acceptable salt or a pharmaceutical composition thereof in combination with the EGFR inhibitor compound or a pharmaceutically acceptable salt or a pharmaceutical composition thereof.

In some aspects of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### BRIEF DESCRIPTION OF THE DRAWINGS

FIG. 1 depicts average tumor volumes (mm³) of NCI-H1975 tumor bearing mice treated with osimertinib and MRTX0902, single agents and in combination.

FIG. 2 depicts average tumor volumes (mm³) of PC-9 tumor bearing mice treated with osimertinib and MRTX0902, single agents and in combination.

### DETAILED DESCRIPTION OF THE INVENTION

The present invention relates to combination therapies for treating SOS1-associated cancers. In particular, the present invention relates to methods of treating cancer in a subject in need thereof, comprising administering to the subject a therapeutically effective amount of a combination of a SOS1 inhibitor or a pharmaceutically acceptable salt or a pharmaceutical composition thereof, and an EGFR inhibitor or a pharmaceutically acceptable salt or a pharmaceutical composition thereof, pharmaceutical compositions comprising therapeutically effective amounts of the inhibitors, kits comprising the compositions and methods of use therefor.

Combinations of the SOS1 inhibitor, or a pharmaceutically acceptable salt or a pharmaceutical composition thereof, with an EGFR inhibitor, or a pharmaceutically acceptable salt or a pharmaceutical composition thereof, synergistically increase the potency of the SOS1 inhibitor against cancer cells that express SOS1 thereby increasing the efficacy and therapeutic index of the SOS1 inhibitor or pharmaceutically acceptable salts thereof.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “EGFR” refers to the epidermal growth factor receptor (EGFR; ErbB-1; HER1 in humans) is a transmembrane protein that is a receptor for members of the epidermal growth factor family (EGF family) of extracellular protein ligands. The epidermal growth factor receptor is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). In many cancer types, mutations affecting EGFR expression or activity could result in cancer.

As used herein, an “EGFR inhibitor” refers to compounds such as osimertinib (TAGRISSO®), gefitinib, erlotinib, afatinib, brigatinib, icotinib, cetuximab and other small and large molecule EGFR inhibitors), or pharmaceutically acceptable salts thereof. These compounds are capable of negatively modulating or inhibiting all or a portion of the enzymatic activity of EGFR, or a mutated form of EGFR.

An “EGFR-associated disease or disorder” as used herein refers to diseases or disorders associated with or mediated by or having a EGFR mutation or over-expression.

As used herein, “SOS1” refers to the Son of sevenless homolog 1 protein encoded by the SOS1 gene that is involved in signaling through RAS pathways.

As used herein, a “SOS1 inhibitor” refers to a compound that is capable of negatively modulating or inhibiting all or a portion of the interaction between KRAS and SOS1.

A “SOS1-associated disease or disorder” as used herein refers to diseases or disorders associated with or mediated by SOS1. A non-limiting example of a SOS1-associated disease or disorder is a SOS1-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12C mutation (e.g., as determined using a regulatory agency-approved, e.g., FDA-approved, assay or kit). In some embodiments, the subject has a tumor that is positive for a KRas G12C mutation (e.g., as determined using a regulatory agency-approved assay or kit). The subject can be a subject with a tumor(s) that is positive for a KRas G12C mutation (e.g., identified as positive using a regulatory agency-approved, e.g., FDA-approved, assay or kit). The subject can be a subject whose tumors have a KRas G12C mutation (e.g., where the tumor is identified as such using a regulatory agency-approved, e.g., FDA-approved, kit or assay). In some embodiments, the subject is suspected of having a KRas G12C gene-associated cancer. In some embodiments, the subject has a clinical record indicating that the subject has a tumor that has a KRas G12C mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W. B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

In some embodiments of any of the methods or uses described herein, an assay is used to determine whether the patient has SOS1 over-expression using a sample (e.g., a biological sample or a biopsy sample such as a paraffin-embedded biopsy sample) from a patient (e.g., a patient suspected of having a SOS1-associated cancer, a patient having one or more symptoms of a SOS1-associated cancer, and/or a patient that has an increased risk of developing a SOS1-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR, quantitative real-time RT-PCR, allele-specific genotyping or ddPCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

As used herein, “an effective amount” of a compound is an amount that is sufficient to negatively modulate or inhibit the activity of the desired target, i.e., SOS1 or EGFR. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of SOS1 or EGFR. Such amount may be administered as a single dosage or may be administered according to a regimen, whereby it is effective.

As used herein, a “therapeutically effective amount of a combination” of two compounds is an amount that together synergistically increases the activity of the combination in comparison to the therapeutically effective amount of each compound in the combination, i.e., more than merely additive. Alternatively, in vivo, the therapeutically effective amount of the combination of a SOS1 inhibitor, or a pharmaceutically acceptable salt or a pharmaceutical composition thereof, and the EGFR inhibitor compound or a pharmaceutically acceptable salt or a pharmaceutical composition thereof, results in an increased duration of overall survival (“OS”) in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the EGFR inhibitor or a pharmaceutically acceptable salt or a pharmaceutical composition thereof, results in an increased duration of progression-free survival (“PFS”) in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the EGFR inhibitor or a pharmaceutically acceptable salt or a pharmaceutical composition thereof, results in increased tumor regression in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the EGFR inhibitor or a pharmaceutically acceptable salt or a pharmaceutical composition thereof, results in increased tumor growth inhibition in subjects relative to treatment with only the EGFR inhibitor. In one embodiment, the therapeutically effective amount of the combination of a SOS1 inhibitor, or a pharmaceutically acceptable salt or a pharmaceutical composition thereof, and the EGFR inhibitor or a pharmaceutically acceptable salt or a pharmaceutical composition thereof, results in an improvement in the duration of stable disease in subjects compared to treatment with only the SOS1 inhibitor, or only the EGFR inhibitor. The amount of each compound in the combination may be the same or different than the therapeutically effective amount of each compound when administered alone as a monotherapy as long as the combination is synergistic. Such amounts may be administered as a single dosage or may be administered according to a regimen, whereby it is effective.

As used herein, “treatment” means any manner in which the symptoms or pathology of a condition, disorder or disease are ameliorated or otherwise beneficially altered. Treatment also encompasses any pharmaceutical use of the compositions herein.

As used herein, “amelioration” of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of an EGFR inhibitor or a SOS1 inhibitor or a pharmaceutically acceptable salt thereof, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

As used herein, the term “contacting” refers to the bringing together of indicated moieties in an in vitro system or an in vivo system. For example, “contacting” a cancer cell includes the administration of a combination provided herein to an individual or subject, such as a human, having KRas G12C, as well as, for example, introducing a combination provided herein into a sample containing a cellular or purified preparation containing KRas G12C.

### EGFR Inhibitor Compounds

In one aspect of the invention, provided herein are methods of treating cancer in a subject in need thereof, comprising administering to the subject a therapeutically effective amount of a combination of a SOS1 inhibitor, or a pharmaceutically acceptable salt or a pharmaceutical composition thereof, and an EGFR inhibitor or a pharmaceutically acceptable salt or a pharmaceutical composition thereof.

In one embodiment, the EGFR inhibitor is:

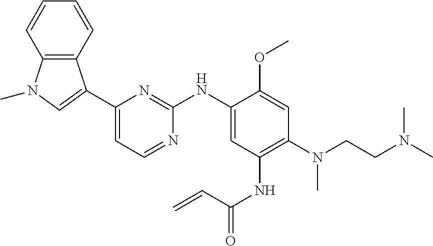

(also referred to as gsimertinib, or TAGRISSO®), or a pharmaceutically acceptable salt thereof.

In one embodiment, the EGFR inhibitor is:

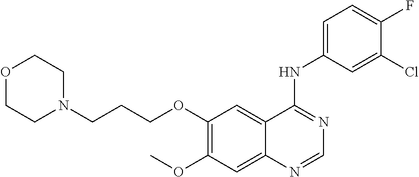 [SMILES: COc1cc2ncnc(Nc3ccc(F)c(Cl)c3)c2cc1OCCCN1CCOCC1]

(also referred to as gefitinib, or IRESSA®), or a pharmaceutically acceptable salt thereof.

In one embodiment, the EGFR inhibitor is:

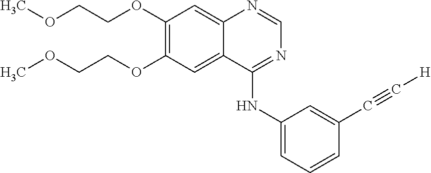 [SMILES: C#Cc1cccc(Nc2ncnc3cc(OCCOC)c(OCCOC)cc23)c1]

(also referred to as erlotinib, or TARCEVA®), or a pharmaceutically acceptable salt thereof.

In one embodiment, the EGFR inhibitor is:

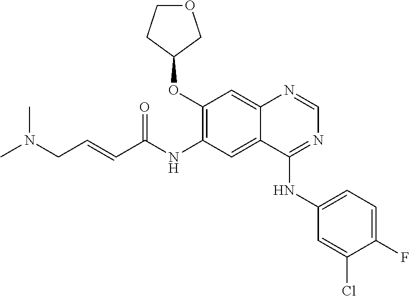 [SMILES: CN(C)C/C=C/C(=O)Nc1cc2c(Nc3ccc(F)c(Cl)c3)ncnc2cc1O[C@H]1CCOC1]

(also referred to as afatinib, or GILOTRIF®), or a pharmaceutically acceptable salt thereof.

In one embodiment, the EGFR inhibitor is:

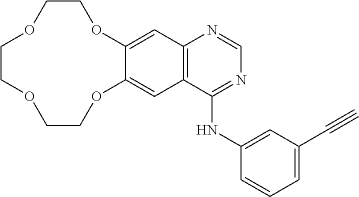 [SMILES: C#Cc1cccc(Nc2ncnc3cc4c(cc23)OCCOCCOCCO4)c1]

(also referred to as icotinib, or CONMANA®), or a pharmaceutically acceptable salt thereof.

In one embodiment, the EGFR inhibitor is the monoclonal antibody cetuximab (also referred to as ERBITUX®).

The EGFR inhibitors used in the methods of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

In one embodiment, the KRas G12C inhibitor compound adagrasib used in the methods include salts of the above compounds, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Methods for manufacturing the KRas G12C inhibitors disclosed herein are generally well known.

### SOS1 Inhibitor Compounds

In one embodiment, the SOS1 inhibitor is a compound selected from (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino) pyrrolidin-1-yl)-4-methylpyrido[3,4-d]pyridazin-1-yl)amino)ethyl)-2-methylbenzonitrile, (R)-3-(1-((6-fluoro-4-methyl-7-(4-methylpiperazin-1-yl) phthalazin-1-yl)amino)ethyl)-2-methylbenzonitrile, or a pharmaceutically acceptable salt thereof; or selected from the compounds described in as described in greater detail herein.

In another embodiment the SOS1 inhibitor is a compound of the formula:

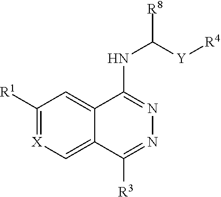

or a pharmaceutically acceptable salt thereof, wherein: R¹ is hydrogen, hydroxyl, C1-C6 alkyl, alkoxy, —N(R⁶)₂,—NR⁶C(O)R⁶,—C(O)N(R⁶)₂,—SO₂alkyl,—SO₂NR⁶alkyl, cycloalkyl, -Q-heterocyclyl, aryl, or heteroaryl, wherein the cycloalkyl, the heterocyclyl, the aryl, and the heteroaryl are each optionally substituted with one or more R²; each Q is independently a bond, O, or NR⁶; X is N or CR⁷; each R² is independently hydroxy, halogen, cyano, hydroxyalkyl, haloalkyl, alkoxy, —N(R⁶)₂,—SO₂alkyl,—NR⁶C(O)C1-C3 alkyl,—C(O)cycloalkyl,—C(O)heretocyclyl or aryl, wherein the cycloalkyl, the heterocyclyl or the aryl are each optionally substituted with one or more R¹¹; R³ is hydrogen, C1-C6 alkyl, alkoxy, —N(R¹⁰)₂, cycloalkyl, haloalkyl, heterocyclyl, aryl, or heteroaryl, wherein the C1-C6 alkyl, the cycloalkyl, the heterocyclyl, the aryl, and the heteroaryl are each optionally substituted with one or more R⁹; Y is a bond or heteroarylene; R⁴ is aryl or heteroaryl, each optionally substituted with one or more R⁵; each R⁵ is independently hydroxy, halogen, cyano, hydroxyalkyl, alkoxy, C1-C3 alkyl, haloalkyl, —N(R⁶)₂,—L-N(R⁶) 2 or —SO₂alkyl; L is C1-C3 alkylene; each R⁶ is independently hydrogen, C1-C3 alkyl, haloalkyl, or cycloalkyl; R⁷ is hydrogen, cyano, or alkoxy; R⁸ is C1-C2 alkyl or halo-C1-C2 alkyl; each R⁹ is independently hydroxy, halogen, amino, cyano, alkoxy, or C1-C3 alkyl; each R¹⁰ is independently hydrogen, C1-C3 alkyl or cycloalkyl; and each R¹¹ is independently C1-C3 alkyl or haloalkyl. These compounds include, but are not limited to, all of the exemplary compounds recited in WO2021/127429, WO2021/173524, WO2022/026465, U.S. provisional patent application 63/213,112 (and corresponding national and international applications and publications) and as described in greater detail herein, including in particular: (R)-2-methyl-3-(1-((4-methyl-7-morpholinopyrido[3,4-d]pyridazin-1-yl)amino)ethyl)benzonitrile.

In another embodiment, the SOS1 inhibitor is a compound selected from:

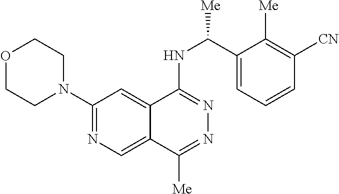 [SMILES: Cc1c(C#N)cccc1[C@@H](C)Nc1nnc(C)c2cnc(N3CCOCC3)cc12]

(R)-2-methyl-3-(1-((4-methyl-7-morpholinopyrido[3,4-d]pyridazin-1-yl)amino)ethyl)benzonitrile,

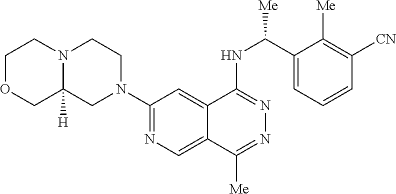 [SMILES: Cc1c(C#N)cccc1[C@@H](C)Nc1nnc(C)c2cnc(N3CCN4CCOC[C@@H]4C3)cc12]

3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile,

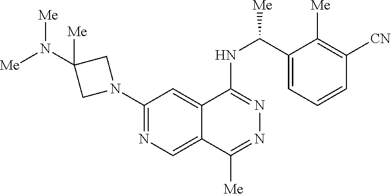 [SMILES: Cc1c(C#N)cccc1[C@@H](C)Nc1nnc(C)c2cnc(N3CC(C)(N(C)C)C3)cc12]

(R)-3-(1-((7-(3-(dimethylamino)-3-methylazetidin-1-yl)-4-methylpyrido[3,4-d]pyridazin-1-yl)amino)ethyl)-2-methylbenzonitrile, and

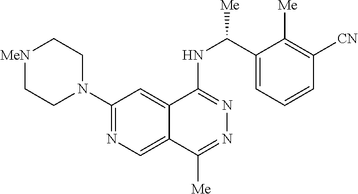 [SMILES: Cc1c(C#N)cccc1[C@@H](C)Nc1nnc(C)c2cnc(N3CCN(C)CC3)cc12]

(R)-2-methyl-3-(1-((4-methyl-7-(4-methylpiperazin-1-yl)pyrido[3,4-d]pyridazin-1-yl)amino)ethyl)benzonitrile,

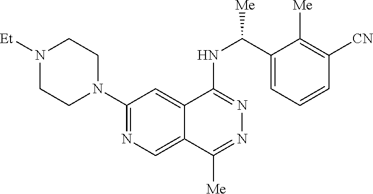 [SMILES: CCN1CCN(c2cc3c(N[C@H](C)c4cccc(C#N)c4C)nnc(C)c3cn2)CC1]

(R)-3-(1-((7-(4-ethylpiperazin-1-yl)-4-methylpyrido[3,4-d]pyridazin-1-yl)amino)ethyl)-2-methylbenzonitrile,

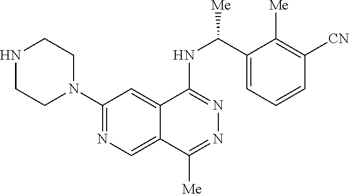 [SMILES: Cc1c(C#N)cccc1[C@@H](C)Nc1nnc(C)c2cnc(N3CCNCC3)cc12]

(R)-2-methyl-3-(1-((4-methyl-7-(piperazin-1-yl)pyrido[3,4-d]pyridazin-1-yl)amino)ethyl)benzonitrile,

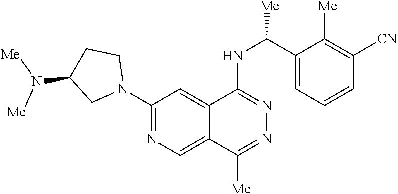 [SMILES: Cc1c(C#N)cccc1[C@@H](C)Nc1nnc(C)c2cnc(N3CC[C@H](N(C)C)C3)cc12]

3-((R)-1-((7-((S)-3-(dimethylamino) pyrrolidin-1-yl)-4-methylpyrido[3,4-d]pyridazin-1-yl)amino)ethyl)-2-methylbenzonitrile, and

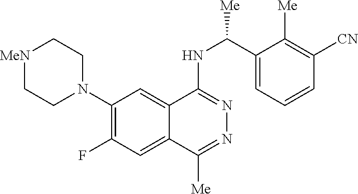 [SMILES: Cc1c(C#N)cccc1[C@@H](C)Nc1nnc(C)c2cc(F)c(N3CCN(C)CC3)cc12]

(R)-3-(1-((6-fluoro-4-methyl-7-(4-methylpiperazin-1-yl) phthalazin-1-yl)amino)ethyl)-2-methylbenzonitrile, and pharmaceutically acceptable salts thereof.

The SOS1 inhibitors used in the methods of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

In one embodiment, the SOS1 inhibitor compound includes its salts, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Methods for manufacturing the SOS1 inhibitors disclosed herein are known. For example, commonly owned applications WO2021/127429, WO2021/173524, WO2022/026465, U.S. provisional patent application 63/213,112 (and corresponding national and international applications and publications) describe general reaction schemes for preparing compounds including adagrasib and also provide detailed synthetic routes for the preparation of these compounds.

### Pharmaceutical Compositions

The SOS1 inhibitors and the EGFR inhibitor or pharmaceutically acceptable salts thereof may be formulated into pharmaceutical compositions.

In another aspect, the invention provides pharmaceutical compositions comprising a SOS1 inhibitor, or a pharmaceutically acceptable salt thereof, and an EGFR inhibitor, or a pharmaceutically acceptable salt thereof and a pharmaceutically acceptable carrier, excipient, or diluent that may be used in the methods disclosed herein. The SOS1 inhibitor, or a pharmaceutically acceptable salt thereof, and EGFRinhibitor, or a pharmaceutically acceptable salt thereof may be independently formulated by any method well known in the art and may be prepared for administration by any route, including, without limitation, parenteral, oral, sublingual, transdermal, topical, intranasal, intratracheal, or intrarectal. In certain embodiments, the SOS1 inhibitor, or a pharmaceutically acceptable salt thereof, and/or the KRas G12C inhibitor, or a pharmaceutically acceptable salt thereof, is/are administered intravenously in a hospital setting.

In one embodiment, administration of one or both therapeutic components may be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salt” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula-NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising a SOS1 inhibitor, or a pharmaceutically acceptable salt or a pharmaceutical composition thereof and an EGFR inhibitor, or a pharmaceutically acceptable salt or a pharmaceutical composition thereof, may be used in the methods of use described herein.

### Co-Adminstration

The SOS1 inhibitor, or a pharmaceutically acceptable salt thereof, and the EGFR inhibitor, or a pharmaceutically acceptable salt thereof, can be formulated into separate or individual dosage forms which can be co-administered one after the other. Another option is that if the route of administration is the same (e.g. oral) two active compounds can be formulated into a single form for co-administration, both methods of co-administration, however, being part of the same therapeutic treatment or regimen.

The pharmaceutical compositions comprising a SOS1 inhibitor, or a pharmaceutically acceptable salt thereof, and/or an EGFR inhibitor, or a pharmaceutically acceptable salt thereof, for use in the methods may be for simultaneous, separate or sequential use. In one embodiment, the SOS1 inhibitor or a pharmaceutically acceptable salt thereof, is administered prior to administration of the EGFR inhibitor or a pharmaceutically acceptable salt thereof. In another embodiment, the SOS1 inhibitor, or a pharmaceutically acceptable salt thereof, is administered after administration of the EGFR inhibitor or a pharmaceutically acceptable salt thereof. In another embodiment, the SOS1 inhibitor, or a pharmaceutically acceptable salt thereof, is administered at about the same time as administration of the EGFR inhibitor compound or a pharmaceutically acceptable salt thereof.

Separate administration of each inhibitor, at different times and by different routes, in some cases would be advantageous. Thus, the components in the combination i.e. the EGFR inhibitor or a pharmaceutically acceptable salt thereof and the SOS1 inhibitor, or a pharmaceutically acceptable salt thereof, need not be necessarily administered at essentially the same time or in any order.

Oncology drugs are typically administered at the maximum tolerated dose (“MTD”), which is the highest dose of drug that does not cause unacceptable side effects. In one embodiment, the EGFR inhibitor, or a pharmaceutically acceptable salt or a pharmaceutical composition thereof and the SOS1 inhibitor, or a pharmaceutically acceptable salt or a pharmaceutical composition thereof, are each dosed at their respective MTDs. In one embodiment, the EGFR inhibitor, or a pharmaceutically acceptable salt or a pharmaceutical composition thereof, is dosed at its MTD and the SOS1 inhibitor, or a pharmaceutically acceptable salt or a pharmaceutical composition thereof, is dosed in an amount less than its MTD. In one embodiment, the EGFR inhibitor, or a pharmaceutically acceptable salt or a pharmaceutical composition thereof, is dosed at an amount less than its MTD and the SOS1 inhibitor, or a pharmaceutically acceptable salt or a pharmaceutical composition thereof, is dosed at its MTD. In one embodiment, the EGFR inhibitor, or a pharmaceutically acceptable salt or a pharmaceutical composition thereof and the SOS1 inhibitor, or a pharmaceutically acceptable salt or a pharmaceutical composition thereof are each dosed at less than their respective MTDs. The administration can be so timed that the peak pharmacokinetic effect of one compound coincides with the peak pharmacokinetic effect of the other.

In one embodiment, a single dose of EGFR inhibitor or a pharmaceutically acceptable salt or a pharmaceutical composition thereof, is administered per day (i.e., in about 24 hour intervals) (i.e., QD). In another embodiment, two doses of the EGFR inhibitor or a pharmaceutically acceptable salt or a pharmaceutical composition thereof, are administered per day (i.e., BID). In another embodiment, three doses of the EGFR inhibitor or a pharmaceutically acceptable salt or a pharmaceutical composition thereof, are administered per day (i.e., TID).

In one embodiment, the SOS1 inhibitor, or a pharmaceutically acceptable salt or a pharmaceutical composition thereof, is administered QD. In another embodiment the SOS1 inhibitor, or a pharmaceutically acceptable salt or a pharmaceutical composition thereof, are administered BID. In another embodiment, the SOS1 inhibitor, or a pharmaceutically acceptable salt or a pharmaceutical composition thereof, of the invention are administered TID.

In one embodiment, a single dose of EGFR inhibitor or a pharmaceutically acceptable salt or a pharmaceutical composition thereof, and SOS1 inhibitor, or a pharmaceutically acceptable salt or a pharmaceutical composition thereof are each administered once daily.

Examples of SOS1 inhibitors suitable for the provided compositions and methods include those mentioned herein, for example (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino) pyrrolidin-1-yl)-4-methylpyrido[3,4-d]pyridazin-1-yl)amino)ethyl)-2-methylbenzonitrile, and (R)-3-(1-((6-fluoro-4-methyl-7-(4-methylpiperazin-1-yl) phthalazin-1-yl)amino)ethyl)-2-methylbenzonitrile.

### Combination Therapies

In one aspect of the invention, provided herein are methods of treating cancer in a subject in need thereof, comprising administering to the subject a therapeutically effective amount of a combination of a SOS1 inhibitor, or a pharmaceutically acceptable salt or a pharmaceutical composition thereof, and the EGFR inhibitor or a pharmaceutically acceptable salt or a pharmaceutical composition thereof. In one embodiment, the cancer is a SOS1-associated cancer. In one embodiment, the SOS1-associated cancer is lung cancer.

In yet another aspect, the invention provides for methods for increasing the sensitivity of a cancer cell to a SOS1 inhibitor, comprising contacting the cancer cell with an effective amount of a combination of the SOS1 inhibitor, such as MRTX0902, or a pharmaceutically acceptable salt or a pharmaceutical composition thereof, and an EGFR inhibitor, such as Osimertinib, or a pharmaceutically acceptable salt or a pharmaceutical composition thereof, wherein the EGFR inhibitor synergistically increases the sensitivity of the cancer cell to the SOS1 inhibitor. In one embodiment, the contacting is in vitro. In one embodiment, the contacting is in vivo.

In one embodiment, the combination therapy comprises a combination of a compound having the formula:

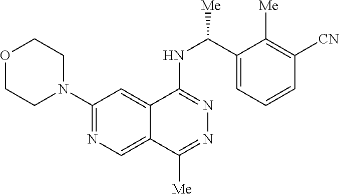 [SMILES: Cc1c(C#N)cccc1[C@@H](C)Nc1nnc(C)c2cnc(N3CCOCC3)cc12]

(also known as MRTX0902) or a pharmaceutically acceptable salt thereof, and an EGFR inhibitor.

In one such embodiment, the EGFR inhibitor is osimertinib.

In one such embodiment, the EGFR inhibitor is gefitinib.

In one such embodiment, the EGFR inhibitor is erlotinib.

In one such embodiment, the EGFR inhibitor is afatinib.

In one such embodiment, the EGFR inhibitor is osimertinib.

In one such embodiment, the EGFR inhibitor is brigatinib.

In one such embodiment, the EGFR inhibitor is icotinib.

In one such embodiment, the EGFR inhibitor is cetuximab.

In one embodiment, the combination therapy comprises a combination osimertinib and a SOS1 inhibitor.

In one such embodiment, the SOS1 inhibitor is:

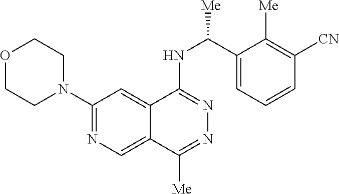 [SMILES: Cc1c(C#N)cccc1[C@@H](C)Nc1nnc(C)c2cnc(N3CCOCC3)cc12]

(R)-2-methyl-3-(1-((4-methyl-7-morpholinopyrido[3,4-d]pyridazin-1-yl)amino)ethyl)benzonitrile (MRTX0902).

In another such embodiment, the SOS1 inhibitor is:

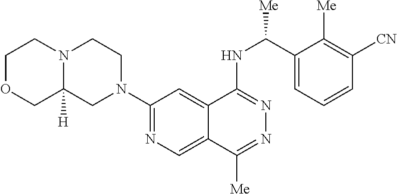 [SMILES: Cc1c(C#N)cccc1[C@@H](C)Nc1nnc(C)c2cnc(N3CCN4CCOC[C@@H]4C3)cc12]

3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile

In yet another such embodiment, the SOS1 inhibitor is:

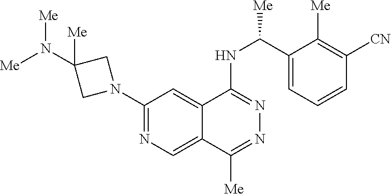 [SMILES: Cc1c(C#N)cccc1[C@@H](C)Nc1nnc(C)c2cnc(N3CC(C)(N(C)C)C3)cc12]

(R)-3-(1-((7-(3-(dimethylamino)-3-methylazetidin-1-yl)-4-methylpyrido[3,4-d]pyridazin-1-yl)amino)ethyl)-2-methylbenzonitrile.

In still another such embodiment, the SOS1 inhibitor is:

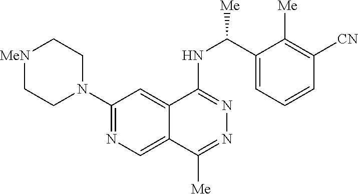 [SMILES: Cc1c(C#N)cccc1[C@@H](C)Nc1nnc(C)c2cnc(N3CCN(C)CC3)cc12]

(R)-2-methyl-3-(1-((4-methyl-7-(4-methylpiperazin-1-yl)pyrido[3,4-d]pyridazin-1-yl)amino)ethyl)benzonitrile.

In another embodiment, the SOS1 inhibitor is:

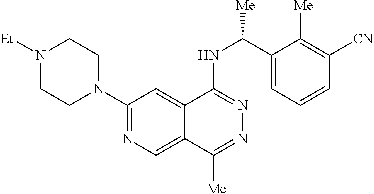 [SMILES: CCN1CCN(c2cc3c(N[C@H](C)c4cccc(C#N)c4C)nnc(C)c3cn2)CC1]

(R)-3-(1-((7-(4-ethylpiperazin-1-yl)-4-methylpyrido[3,4-d]pyridazin-1-yl)amino)ethyl)-2-methylbenzonitrile.

In another embodiment, the SOS1 inhibitor is:

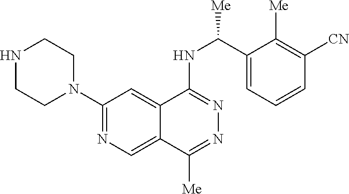 [SMILES: Cc1c(C#N)cccc1[C@@H](C)Nc1nnc(C)c2cnc(N3CCNCC3)cc12]

(R)-2-methyl-3-(1-((4-methyl-7-(piperazin-1-yl)pyrido[3,4-d]pyridazin-1-yl)amino)ethyl)benzonitrile.

In another embodiment, the SOS1 inhibitor is:

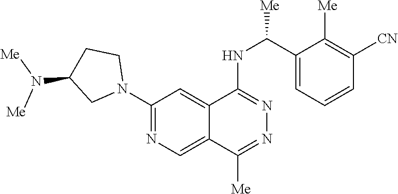 [SMILES: Cc1c(C#N)cccc1[C@@H](C)Nc1nnc(C)c2cnc(N3CC[C@H](N(C)C)C3)cc12]

3-((R)-1-((7-((S)-3-(dimethylamino) pyrrolidin-1-yl)-4-methylpyrido[3,4-d]pyridazin-1-yl)amino)ethyl)-2-methylbenzonitrile.

In another embodiment, the SOS1 inhibitor is:

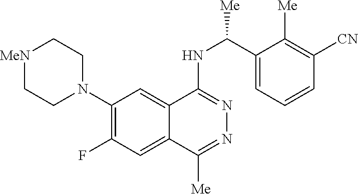 [SMILES: Cc1c(C#N)cccc1[C@@H](C)Nc1nnc(C)c2cc(F)c(N3CCN(C)CC3)cc12]

(R)-3-(1-((6-fluoro-4-methyl-7-(4-methylpiperazin-1-yl) phthalazin-1-yl)amino)ethyl)-2-methylbenzonitrile.

The methods described herein are designed to inhibit undesired cellular proliferation resulting from enhanced EGFR activity within the cell. The degree of inhibitory activity of the SOS1 inhibitor-EGFR inhibitor combination in cells may be monitored, for example, by measuring cell viability and functional inhibition of both RAF/MEK/ERK and PI3K/AKT effector pathway signaling (amounts of phosphorylated ERK and AKT, respectively) to assess the effectiveness of treatment and dosages may be adjusted accordingly by the attending medical practitioner.

The compositions and methods provided herein may be used for the treatment of a SOS1-associated cancer in a subject in need thereof, comprising administering to said subject a therapeutically effective amount of a combination of a SOS1 inhibitor, or a pharmaceutically acceptable salt or a pharmaceutical composition thereof, and an EGFR inhibitor or a pharmaceutically acceptable salt or a pharmaceutical composition thereof, wherein the EGFR inhibitor synergistically increases the sensitivity of the SOS1-associated cancer to the SOS1 inhibitor. In one embodiment, the SOS1 associated cancer is a cancer with genetic alterations of the MAPK pathway. In one embodiment, the SOS1 associated cancer is a cancer mediated by SOS1. In one embodiment, the SOS1-associated cancer is selected from the group consisting of leukemia, uterine cancer, lung cancer, colorectal cancer and pancreatic cancer. In one embodiment, leukemia is acute myeloid leukemia (AML). In one embodiment, lung cancer is lung adenocarcinoma. In one embodiment, lung cancer is non-small cell lung cancer. In one embodiment, the pancreatic cancer is ductal carcinoma of the pancreas.

In one embodiment, the therapeutically effective amount of the combination of a SOS1 inhibitor, or a pharmaceutically acceptable salt or a pharmaceutical composition thereof, and an EGFRinhibitor or a pharmaceutically acceptable salt or a pharmaceutical composition thereof, results in an increased duration of overall survival (“OS”) in subjects relative to treatment with only the SOS1 inhibitor. In one embodiment, the therapeutically effective amount of the combination of a SOS1 inhibitor, or a pharmaceutically acceptable salt or a pharmaceutical composition thereof, and an EGFR inhibitor or a pharmaceutically acceptable salt or a pharmaceutical composition thereof, results in an increased duration of progression-free survival (“PFS”) in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the EGFR inhibitor or a pharmaceutically acceptable salt or a pharmaceutical composition thereof, results in increased tumor regression in subjects relative to treatment with only the SOS1 inhibitor. In one embodiment, the therapeutically effective amount of the combination of a SOS1 inhibitor, or a pharmaceutically acceptable salt or a pharmaceutical composition thereof, and EGFR inhibitor or a pharmaceutically acceptable salt or a pharmaceutical composition thereof, results in increased tumor growth inhibition in subjects relative to treatment with only the SOS1 inhibitor. In one embodiment, the therapeutically effective amount of the combination of a SOS1 inhibitor, or a pharmaceutically acceptable salt or a pharmaceutical composition thereof, and the EGFR inhibitor or a pharmaceutically acceptable salt or a pharmaceutical composition thereof, results in an improvement in the duration of stable disease in subjects compared to treatment with only the SOS1 inhibitor.

In one embodiment, the SOS1 inhibitor is selected from (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino) pyrrolidin-1-yl)-4-methylpyrido[3,4-d]pyridazin-1-yl)amino)ethyl)-2-methylbenzonitrile, (R)-3-(1-((6-fluoro-4-methyl-7-(4-methylpiperazin-1-yl) phthalazin-1-yl)amino)ethyl)-2-methylbenzonitrile, or a pharmaceutically acceptable salt thereof.

In another embodiment, the SOS1 inhibitor, or a pharmaceutically acceptable salt or a pharmaceutical composition thereof, is administered in combination with the EGFR inhibitor, or a pharmaceutically acceptable salt or a pharmaceutical composition thereof, once disease progression has been observed for SOS1 monotherapy, in which the combination therapy results in enhanced clinical benefit for the patient by increasing OS, PFS, tumor regression, tumor growth inhibition or the duration of stable disease in the patient.

In one embodiment, the therapeutic combination comprises therapeutically effective amounts of osimertinib and (R)-2-methyl-3-(1-((4-methyl-7-morpholinopyrido[3,4-d]pyridazin-1-yl)amino)ethyl)benzonitrile, or a pharmaceutically acceptable salt thereof.

In another embodiment, the therapeutic combination comprises therapeutically effective amounts of osimertinib and 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile, or a pharmaceutically acceptable salt thereof.

In another embodiment, the therapeutic combination comprises therapeutically effective amounts of osimertinib and (R)-3-(1-((7-(3-(dimethylamino)-3-methylazetidin-1-yl)-4-methylpyrido[3,4-d]pyridazin-1-yl)amino)ethyl)-2-methylbenzonitrile, or a pharmaceutically acceptable salt thereof.

In another embodiment, the therapeutic combination comprises therapeutically effective amounts of osimertinib and (R)-2-methyl-3-(1-((4-methyl-7-(4-methylpiperazin-1-yl)pyrido[3,4-d]pyridazin-1-yl)amino)ethyl)benzonitrile, or a pharmaceutically acceptable salt thereof.

In another embodiment, the therapeutic combination comprises therapeutically effective amounts of osimertinib and (R)-3-(1-((7-(4-ethylpiperazin-1-yl)-4-methylpyrido[3,4-d]pyridazin-1-yl)amino)ethyl)-2-methylbenzonitrile, or a pharmaceutically acceptable salt thereof.

In another embodiment, the therapeutic combination comprises therapeutically effective amounts of osimertinib and (R)-2-methyl-3-(1-((4-methyl-7-(piperazin-1-yl)pyrido[3,4-d]pyridazin-1-yl)amino)ethyl)benzonitrile, or a pharmaceutically acceptable salt thereof.

In another embodiment, the therapeutic combination comprises therapeutically effective amounts of osimertinib and 3-((R)-1-((7-((S)-3-(dimethylamino) pyrrolidin-1-yl)-4-methylpyrido[3,4-d]pyridazin-1-yl)amino)ethyl)-2-methylbenzonitrile, or a pharmaceutically acceptable salt thereof.

In another embodiment, the therapeutic combination comprises therapeutically effective amounts of osimertinib and (R)-3-(1-((6-fluoro-4-methyl-7-(4-methylpiperazin-1-yl) phthalazin-1-yl)amino)ethyl)-2-methylbenzonitrile, or a pharmaceutically acceptable salt thereof.

The compositions and methods provided herein may be used for the treatment of a wide variety of cancers including tumors such as lung, colorectal, pancreas, prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

Also provided herein is a method for treating cancer in a subject in need thereof, the method comprising (a) determining that cancer is associated with SOS1 over-expression (e.g., as determined using a regulatory agency-approved, e.g., FDA-approved, assay or kit); and (b) administering to the patient a therapeutically effective amount of a combination of a SOS1 inhibitor, or a pharmaceutically acceptable salt or a pharmaceutical composition thereof, and an EGFRinhibitor or a pharmaceutically acceptable salt or a pharmaceutical composition thereof, wherein the EGFR inhibitor synergistically increases the sensitivity of the SOS1-associated cancer to the SOS1 inhibitor. In one embodiment, the EGFR inhibitor is selected from osimertinib, gefitinib, erlotinib, afatinib, brigatinib, icotinib, cetuximab and other small and large molecule EGFR inhibitors, and the SOS1 inhibitor is selected from: (R)-2-methyl-3-(1-((4-methyl-7-morpholinopyrido[3,4-d]pyridazin-1-yl)amino)ethyl)benzonitrile, 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile, (R)-3-(1-((7-(3-(dimethylamino)-3-methylazetidin-1-yl)-4-methylpyrido[3,4-d]pyridazin-1-yl)amino)ethyl)-2-methylbenzonitrile, (R)-2-methyl-3-(1-((4-methyl-7-(4-methylpiperazin-1-yl)pyrido[3,4-d]pyridazin-1-yl)amino)ethyl)benzonitrile, (R)-3-(1-((7-(4-ethylpiperazin-1-yl)-4-methylpyrido[3,4-d]pyridazin-1-yl)amino)ethyl)-2-methylbenzonitrile, (R)-2-methyl-3-(1-((4-methyl-7-(piperazin-1-yl)pyrido[3,4-d]pyridazin-1-yl)amino)ethyl)benzonitrile, 3-((R)-1-((7-((S)-3-(dimethylamino) pyrrolidin-1-yl)-4-methylpyrido[3,4-d]pyridazin-1-yl)amino)ethyl)-2-methylbenzonitrile, (R)-3-(1-((6-fluoro-4-methyl-7-(4-methylpiperazin-1-yl) phthalazin-1-yl)amino)ethyl)-2-methylbenzonitrile, or a pharmaceutically acceptable salt thereof.

In one embodiment, the therapeutic combination comprises therapeutically effective amounts of (R)-2-methyl-3-(1-((4-methyl-7-morpholinopyrido[3,4-d]pyridazin-1-yl)amino)ethyl)benzonitrile or a pharmaceutically acceptable salt thereof.

In a further embodiment, the therapeutic combination comprises therapeutically effective amounts of 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile or a pharmaceutically acceptable salt thereof.

In a further embodiment, the therapeutic combination comprises therapeutically effective amounts of (R)-3-(1-((7-(3-(dimethylamino)-3-methylazetidin-1-yl)-4-methylpyrido[3,4-d]pyridazin-1-yl)amino)ethyl)-2-methylbenzonitrile or a pharmaceutically acceptable salt thereof.

In a further embodiment, the therapeutic combination comprises therapeutically effective amounts of (R)-2-methyl-3-(1-((4-methyl-7-(4-methylpiperazin-1-yl)pyrido[3,4-d]pyridazin-1-yl)amino)ethyl)benzonitrile or a pharmaceutically acceptable salt thereof.

In a further embodiment, the therapeutic combination comprises therapeutically effective amounts of (R)-3-(1-((7-(4-ethylpiperazin-1-yl)-4-methylpyrido[3,4-d]pyridazin-1-yl)amino)ethyl)-2-methylbenzonitrile or a pharmaceutically acceptable salt thereof.

In a further embodiment, the therapeutic combination comprises therapeutically effective amounts of (R)-2-methyl-3-(1-((4-methyl-7-(piperazin-1-yl)pyrido[3,4-d]pyridazin-1-yl)amino)ethyl)benzonitrile or a pharmaceutically acceptable salt thereof.

In a further embodiment, the therapeutic combination comprises therapeutically effective amounts of 3-((R)-1-((7-((S)-3-(dimethylamino) pyrrolidin-1-yl)-4-methylpyrido[3,4-d]pyridazin-1-yl)amino)ethyl)-2-methylbenzonitrile or a pharmaceutically acceptable salt thereof.

In a further embodiment, the therapeutic combination comprises therapeutically effective amounts of (R)-3-(1-((6-fluoro-4-methyl-7-(4-methylpiperazin-1-yl) phthalazin-1-yl)amino)ethyl)-2-methylbenzonitrile or a pharmaceutically acceptable salt thereof.

In one embodiment, the SOS1 inhibitor, the EGFR inhibitor, or both, is/are administered as a tablet or capsule during the period of time. In one embodiment, the tablet or capsule formulation of the SOS1 inhibitor and/or the EGR inhibitor comprises one or more of: about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg and about 2000 mg. In one embodiment, the SOS1 inhibitor and/or the EGFR inhibitor is orally administered once a day (QD) on a daily basis during a period of time. In one embodiment SOS1 inhibitor and/or the EGFR inhibitor is orally administered twice a day (BID) on a daily basis during a period of time.

In one embodiment, the SOS1 inhibitor and/or the EGFR inhibitor is/are orally administered in the amount of about 20 mg to about 500 mg (e.g., about 20 mg to about 480 mg, about 20 mg to about 460 mg, about 20 mg to about 440 mg, about 20 mg to about 420 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 500 mg, about 40 mg to about 480 mg, about 40 mg to about 460 mg, about 40 mg to about 440 mg, about 40 mg to about 420 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 500 mg, about 60 mg to about 480 mg, about 60 mg to about 460 mg, about 60 mg to about 440 mg, about 60 mg to about 420 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 500 mg, about 80 mg to about 480 mg, about 80 mg to about 460 mg, about 80 mg to about 440 mg, about 80 mg to about 420 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 500 mg, about 100 mg to about 480 mg, about 100 mg to about 460 mg, about 100 mg to about 440 mg, about 100 mg to about 420 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 500 mg, about 120 mg to about 480 mg, about 120 mg to about 460 mg, about 120 mg to about 440 mg, about 120 mg to about 420 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 500 mg, about 140 mg to about 480 mg, about 140 mg to about 460 mg, about 140 mg to about 440 mg, about 140 mg to about 420 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 500 mg, about 160 mg to about 480 mg, about 160 mg to about 460 mg, about 160 mg to about 440 mg, about 160 mg to about 420 mg, about 160 mg to about 400 mg, about 160 mg to about 380 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 500 mg, about 180 mg to about 480 mg, about 180 mg to about 460 mg, about 180 mg to about 440 mg, about 180 mg to about 420 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 500 mg, about 200 mg to about 480 mg, about 200 mg to about 460 mg, about 200 mg to about 440 mg, about 200 mg to about 420 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 500 mg, about 220 mg to about 480 mg, about 220 mg to about 460 mg, about 220 mg to about 440 mg, about 220 mg to about 420 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 500 mg, about 240 mg to about 480 mg, about 240 mg to about 460 mg, about 240 mg to about 440 mg, about 240 mg to about 420 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 500 mg, about 260 mg to about 480 mg, about 260 mg to about 460 mg, about 260 mg to about 440 mg, about 260 mg to about 420 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 500 mg, about 280 mg to about 480 mg, about 280 mg to about 460 mg, about 280 mg to about 440 mg, about 280 mg to about 420 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 500 mg, about 300 mg to about 480 mg, about 300 mg to about 460 mg, about 300 mg to about 440 mg, about 300 mg to about 420 mg, about 300 mg to about 400 mg, about 300 mg to about 380 mg, about 300 mg to about 360 mg, about 300 mg to about 340 mg, about 300 mg to about 320 mg, about 320 mg to about 500 mg, about 320 mg to about 480 mg, about 320 mg to about 460 mg, about 320 mg to about 440 mg, about 320 mg to about 420 mg, about 320 mg to about 400 mg, about 320 mg to about 380 mg, about 320 mg to about 360 mg, about 320 mg to about 340 mg, about 340 mg to about 500 mg, about 340 mg to about 480 mg, about 340 mg to about 460 mg, about 340 mg to about 440 mg, about 340 mg to about 420 mg, about 340 mg to about 400 mg, about 340 mg to about 380 mg, about 340 mg to about 360 mg, about 360 mg to about 500 mg, about 360 mg to about 480 mg, about 360 mg to about 460 mg, about 360 mg to about 440 mg, about 360 mg to about 420 mg, about 360 mg to about 400 mg, about 360 mg to about 380 mg, about 380 mg to about 500 mg, about 380 mg to about 480 mg, about 380 mg to about 460 mg, about 380 mg to about 440 mg, about 380 mg to about 420 mg, about 380 mg to about 400 mg, about 400 mg to about 500 mg, about 400 mg to about 480 mg, about 400 mg to about 460 mg, about 400 mg to about 440 mg, about 400 mg to about 420 mg, about 420 mg to about 500 mg, about 420 mg to about 480 mg, about 420 mg to about 460 mg, about 420 mg to about 440 mg, about 440 mg to about 500 mg, about 440 mg to about 480 mg, about 440 mg to about 460 mg, about 460 mg to about 500 mg, about 460 mg to about 480 mg, about 480 mg to about 500 mg, about 25, about 50, about 75, about 100, about 150, about 200, about 250, about 300, about 350, about 400, about 450, or about 500 mg) over a period of time. In one embodiment, the SOS1 inhibitor and/or the EGFR inhibitor is/are orally administered twice a day (BID) on a daily basis during a period of time. In one embodiment adagrasib is orally administered twice a day (BID) on a daily basis during a period of time.

In one embodiment, the combination therapy comprises oral administration of the SOS1 inhibitor and/or the EGFR inhibito, each independently once or twice a day on a daily basis (during a period of time), e.g., in an amount of about 10 mg to about 400 mg (e.g., about 10 mg to about 380 mg, about 10 mg to about 360 mg, about 10 mg to about 340 mg, about 10 mg to about 320 mg, about 10 mg to about 300 mg, about 10 mg to about 280 mg, about 10 mg to about 260 mg, about 10 mg to about 240 mg, about 10 mg to about 220 mg, about 10 mg to about 200 mg, about 10 mg to about 180 mg, about 10 mg to about 160 mg, about 10 mg to about 140 mg, about 10 mg to about 120 mg, about 10 mg to about 100 mg, about 10 mg to about 80 mg, about 10 mg to about 60 mg, about 10 mg to about 40 mg, about 10 mg to about 20 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 400 mg, about 160 mg to about 380 mg, about 160 mg to about 360 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 400 mg, about 300 mg to about 380 mg, about 300 mg to about 360 mg, about 300 mg to about 340 mg, about 300 mg to about 320 mg, about 320 mg to about 400 mg, about 320 mg to about 380 mg, about 320 mg to about 360 mg, about 340 mg to about 360 mg, about 340 mg to about 400 mg, about 340 mg to about 380 mg, about 340 mg to about 360 mg, about 360 mg to about 400 mg, about 360 mg to about 380 mg, about 380 mg to about 400 mg, about 100 mg, about 200 mg, about 300 mg, or about 400 mg), . . . . In one embodiment, the KRas G12C inhibitor adagrasib or a pharmaceutically acceptable salt or a pharmaceutical composition thereof is orally administered once daily. In another embodiment, the KRas G12C inhibitor, or a pharmaceutically acceptable salt or a pharmaceutical composition thereof, is orally administered twice daily.

One skilled in the art will recognize that, both in vivo and in vitro trials using suitable, known and generally accepted cell and/or animal models are predictive of the ability of a test compound of the combination or the combination to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### Synergy

In one embodiment, the addition of an EGFR inhibitor, or a pharmaceutically acceptable salt or a pharmaceutical composition thereof, synergistically increases the activity of the SOS1inhibitor compound, for instance MRTX0902 or a pharmaceutically acceptable salt or a pharmaceutical composition thereof, against cancer or cancer cell lines over-expressing SOS1. Any method for determining whether two compounds exhibit synergy may be used for determining the synergistic effect of the combination.

Several mathematical models have been developed to determine whether two compounds act synergistically, i.e., beyond a mere additive effect. For instance, Loewe Additivity (Loewe (1928) Physiol. 27:47-187), Bliss Independence (Bliss (1939) Ann. Appl. Biol. 26:585-615), Highest Single Agent, ZIP (Yadav et al (2015) Comput Struct Biotech J 13:504-513) and other models (Chou & Talalay (1984) Adv Enzyme Regul 22:27-55. #6382953; and Greco et al. (1995) Pharmacol Rev 47 (2): 331-85. #7568331) are well known models in the pharmaceutical industry and may be used to calculate a “synergy score” that indicates whether synergy was detected and the magnitude of such synergy. Combining these synergy scores produces a composite synergy score which may be used to evaluate and characterize an EGFR inhibitor such as osimertinib and a SOS1 inhibitor such as MRTX0902.

In general, the mathematical models use data obtained from single agent values to determine the predicted additive effect of the combination which is compared to the observed effect for the combination. If the observed effect is greater than the predicted effect, the combination is deemed to be synergistic. For example, the Bliss independence model compares the observed combination response (Y_{O}) with the predicted combination response (Y_{P}), which was obtained based on the assumption that there is no effect from drug-drug interactions. Typically, the combination effect is declared synergistic if Y_{O} is greater than Y_{P}.

In some embodiments, “synergistic effect” as used herein refers to combination of an EGFR inhibitor or a pharmaceutically acceptable salt thereof, and a SOS1 inhibitor or a pharmaceutically acceptable salt thereof producing an effect, for example, any of the beneficial or desired results including clinical results or endpoints as described herein, which is greater than the sum of the effect observed when a compound such as one described in the SOS1 patent applications recited herein, for instance MRTX0902, and an EGFR inhibitor or a pharmaceutically acceptable salt thereof, for instance osimertinib, are administered alone. acceptable salt thereof.

In one embodiment, the synergistic therapeutic combination comprises therapeutically effective amounts of (R)-2-methyl-3-(1-((4-methyl-7-morpholinopyrido[3,4-d]pyridazin-1-yl)amino)ethyl)benzonitrile and osimertinib. In one embodiment, the synergistic therapeutic combination comprises therapeutically effective amounts of 3-((R)-1-((7-((S)-hexahydropyrazino[2,1-c][1,4]oxazin-8 (1H)-yl)-4-methylpyrido[3,4-d]pyridazin-1-yl)amino)ethyl)-2-methylbenzonitrile and osimertinib. In one embodiment, the synergistic therapeutic combination comprises therapeutically effective amounts of (R)-3-(1-((7-(3-(dimethylamino)-3-methylazetidin-1-yl)-4-methylpyrido[3,4-d]pyridazin-1-yl)amino)ethyl)-2-methylbenzonitrile and osimertinib. In one embodiment, the synergistic therapeutic combination comprises therapeutically effective amounts of (R)-2-methyl-3-(1-((4-methyl-7-(4-methylpiperazin-1-yl)pyrido[3,4-d]pyridazin-1-yl)amino)ethyl)benzonitrile and osimertinib. In one embodiment, the synergistic therapeutic combination comprises therapeutically effective amounts of (R)-3-(1-((7-(4-ethylpiperazin-1-yl)-4-methylpyrido[3,4-d]pyridazin-1-yl)amino)ethyl)-2-methylbenzonitrile, and osimertinib. In one embodiment, the synergistic therapeutic combination comprises therapeutically effective amounts of (R)-2-methyl-3-(1-((4-methyl-7-(piperazin-1-yl)pyrido[3,4-d]pyridazin-1-yl)amino)ethyl)benzonitrile, and osimertinib. In one embodiment, the synergistic therapeutic combination comprises therapeutically effective amounts of 3-((R)-1-((7-((S)-3-(dimethylamino) pyrrolidin-1-yl)-4-methylpyrido[3,4-d]pyridazin-1-yl)amino)ethyl)-2-methylbenzonitrile, and osimertinib. In one embodiment, the synergistic therapeutic combination comprises therapeutically effective amounts of (R)-3-(1-((6-fluoro-4-methyl-7-(4-methylpiperazin-1-yl) phthalazin-1-yl)amino)ethyl)-2-methylbenzonitrile and osimertinib.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### Kits

The present invention also relates to a kit comprising a SOS1 inhibitor, or a pharmaceutically acceptable salt or a pharmaceutical composition thereof (for example, MRTX0902), and an EGFR inhibitor or a pharmaceutically acceptable salt or a pharmaceutical composition thereof (for example osimertinib). Also provided is a kit comprising such a SOS1 inhibitor, or a pharmaceutically acceptable salt or a pharmaceutical composition thereof, and such an EGFR inhibitor or a pharmaceutically acceptable salt or a pharmaceutical composition thereof, for use in treating a hematological cancer.

In a related aspect, the invention provides a kit containing a dose of a SOS1 inhibitor, or a pharmaceutically acceptable salt or a pharmaceutical composition thereof, and dose of an EGFR inhibitor or a pharmaceutically acceptable salt or a pharmaceutical composition thereof, in an amount effective to inhibit proliferation of cancer cells, particularly SOS1 over-expressing cancer cells, in a subject. The kit in some cases includes an insert with instructions for administration of the a SOS1 inhibitor, or a pharmaceutically acceptable salt or a pharmaceutical composition thereof, and the EGFR inhibitor or a pharmaceutically acceptable salt or a pharmaceutical composition thereof. The insert may provide a user with one set of instructions for using the a SOS1 inhibitor, or a pharmaceutically acceptable salt or a pharmaceutical composition thereof, in combination with the EGFR inhibitor or a pharmaceutically acceptable salt or a pharmaceutical composition thereof.

The following Examples are intended to illustrate further certain embodiments of the invention and are not intended to limit the scope of the invention.

### Example A

### In Vivo Models for Examining SOS1 Inhibitor-EGFR Inhibitor Combinations

Immunocompromised nude/nude mice are inoculated in the right hind flank with NCI-H1975 cells harboring EGFR L585R/T790M mutations. When tumor volumes reach between 200-400 mm³ in size, the mice are divided into four groups of 4-12 mice each. The first group is administered vehicle only. The second and third group is administered a single agent dose of the EGFR inhibitor osimertinib at a 2.5 mg/kg or 5 mg/kg concentration that yields less than maximal biological effect and does not result in complete tumor regression. The fourth group is administered a single agent dose of the SOS1 inhibitor MRTX0902 at a 50 mg/kg concentration that yields a maximal biological effect but does not result in complete tumor regression. The fourth and fifth groups are administered the single agent dose of the EGFR inhibitor in combination with the single agent dose of the SOS1 inhibitor. The treatment period was 21 days. Tumor volumes are measured using a caliper every two-three days and tumor volumes are calculated by the formula: 0.5×(Length×Width)2. A greater degree of tumor growth inhibition for the combination in this model demonstrates that the combination therapy is likely to have a clinically meaningful benefit to treated subjects relative to treatment with only an EGFR inhibitor.

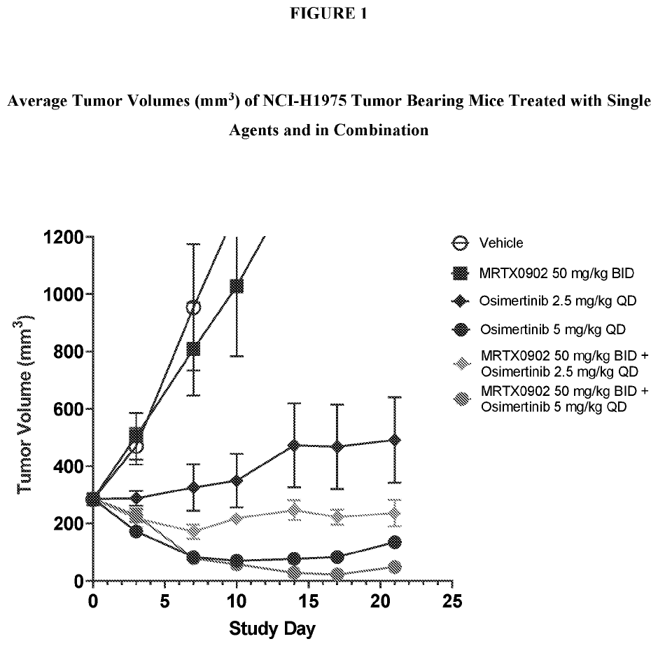

30 nude/nude mice were inoculated in the right hind limb with 5×10⁶ NCI-H1975 cells. When tumor volume reached ˜200-400 mm³ (Study Day 0), 5 mice in each of the six groups were administered p.o. daily for 21 days: vehicle only (0.5% MC (4000 cps)/0.2% Tween 80 in water), 2.5 mg/kg or 5 mg/kg of EGFR inhibitor osimertinib (5% DMSO, 45% PEG400, 50% water), 50 mg/kg of the SOS1 inhibitor (R)-2-methyl-3-(1-((4-methyl-7-morpholinopyrido[3,4-d]pyridazin-1-yl)amino)ethyl)benzonitrile (MRTX0902) (0.5% MC (4000 cps)/0.2% Tween80 in water), or 50 mg/kg MRTX0902 and either 2.5 mg/kg or 5 mg/kg osimertinib. Tumor volumes, measured at pre-specified days, for the five mice per group were averaged and are reported for NCI-H1975 cells in Table 1 and FIG. 1.

TABLE 1
Average Tumor Volumes (mm³) of NCI-H1975 Tumor Bearing
Mice Treated with Single Agents and in Combination

| | | | | | MRTX0902 + | MRTX0902 + |
|---|---|---|---|---|---|---|
| Study | | Osimertinib | Osimertinib | | Osimertinib | Osimertinib |
| Day | Vehicle | 2.5 mg/kg QD | 5 mg/kg QD | MRTX0902 | 2.5 mg/kg | 5 mg/kg |
| 0 | 284 | 286 | 286 | 285 | 287 | 287 |
| 3 | 470 | 288 | 173 | 504 | 219 | 228 |
| 7 | 954 | 325 | 83 | 808 | 172 | 78 |
| 10 | 1293 | 349 | 70 | 1028 | 218 | 58 |
| 14 | 1617 | 473 | 77 | 1387 | 247 | 28 |
| 17 | 1965 | 468 | 83 | 1527 | 222 | 22 |
| 21 | | 491 | 135 | 1372 | 237 | 48 |

As shown in Table 1 and FIG. 1, the administration of osimertinib at 2.5 mg/kg or 5 mg/kg as a single agent resulted in 89% tumor growth and −71% tumor regression at day 17, respectively. The administration of MRTX0902 at 50 mg/kg BID as a single agent resulted in 26% tumor growth inhibition. The combination of the SOS1 inhibitor MRTX0902 and osimertinib at 2.5 mg/kg or 5 mg/kg resulted in −22.6% and −92.3% tumor regression at day 17, respectively.

These results demonstrate that the combination therapy resulted in greater amount of tumor growth inhibition compared to either single agent alone demonstrating enhanced in vivo anti-tumor efficacy of the combination against EGFR L858R T790M expressing cancer.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

### Example B

### In Vivo Models for Examining SOS1 Inhibitor-EGFR Inhibitor Combinations

Immunocompromised NOD/SCID mice are inoculated in the right hind flank with PC9 cells harboring EGFR exon 19del (E746_A750del) mutation. When tumor volumes reach between 100-150 mm³ in size, the mice are divided into four groups of 4-12 mice each. The first group is administered vehicle only. The second and third group is administered a single agent dose of the EGFR inhibitor osimertinib at a 2.5 mg/kg or 5 mg/kg concentration that yields less than maximal biological effect and does not result in complete tumor regression. The fourth group is administered a single agent dose of the SOS1 inhibitor MRTX0902 at a 50 mg/kg concentration that yields a maximal biological effect but does not result in complete tumor regression. The fourth and fifth groups are administered the single agent dose of the EGFR inhibitor in combination with the single agent dose of the SOS1 inhibitor. The treatment period was 27 days. Tumor volumes are measured using a caliper every two-three days and tumor volumes are calculated by the formula: 0.5×(Length×Width) 2. A greater degree of tumor growth inhibition for the combination in this model demonstrates that the combination therapy is likely to have a clinically meaningful benefit to treated subjects relative to treatment with only an EGFR inhibitor.

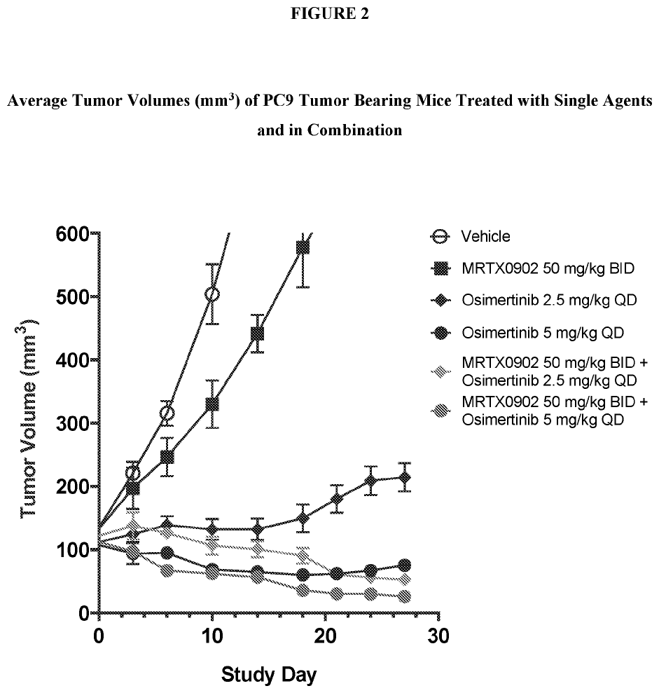

30 NOD/SCID mice were inoculated in the right hind limb with 5×10⁶ PC9 cells. When tumor volume reached ˜100-150 mm³ (Study Day 0), 5 mice in each of the six groups were administered p.o. daily for 27 days: vehicle only (0.5% MC (4000 cps)/0.2% Tween 80 in water), 2.5 mg/kg or 5 mg/kg of EGFR inhibitor osimertinib (5% DMSO, 45% PEG400, 50% water), 50 mg/kg of the SOS1 inhibitor (R)-2-methyl-3-(1-((4-methyl-7-morpholinopyrido[3,4-d]pyridazin-1-yl)amino)ethyl)benzonitrile (MRTX0902) (0.5% MC (4000 cps)/0.2% Tween80 in water), or 50 mg/kg MRTX0902 and either 2.5 mg/kg or 5 mg/kg osimertinib. Tumor volumes, measured at pre-specified days, for the five mice per group were averaged and are reported for PC9 cells in Table 2 and FIG. 2.

TABLE 2
Average Tumor Volumes (mm³) of PC9 Tumor Bearing Mice
Treated with Single Agents and in Combination

| | | | | | MRTX0902 + | MRTX0902 + |
|---|---|---|---|---|---|---|
| Study | | Osimertinib | Osimertinib | | Osimertinib | Osimertinib |
| Day | Vehicle | 2.5 mg/kg QD | 5 mg/kg QD | MRTX0902 | 2.5 mg/kg | 5 mg/kg |
| −1 | 106 | 108 | 111 | 112 | 117 | 117 |
| 3 | 221 | 124 | 94 | 197 | 138 | 97 |
| 6 | 315 | 139 | 95 | 247 | 126 | 67 |
| 10 | 504 | 132 | 69 | 330 | 106 | 62 |
| 14 | 761 | 132 | 65 | 442 | 100 | 57 |
| 18 | 924 | 150 | 60 | 578 | 90 | 36 |
| 21 | 1180 | 180 | 62 | 661 | 61 | 30 |
| 24 | 1530 | 209 | 67 | 755 | 56 | 30 |
| 27 | 1610 | 214 | 75 | 798 | 52 | 26 |

As shown in Table 2 and FIG. 2, the administration of osimertinib at 2.5 mg/kg or 5 mg/kg as a single agent resulted in 92.8% tumor growth and −32.5% tumor regression at day 27, respectively. The administration of MRTX0902 at 50 mg/kg BID resulted in 54% tumor growth inhibition. The combination of the SOS1 inhibitor MRTX0902 and osimertinib at 2.5 mg/kg or 5 mg/kg resulted in-55.3% and −78% tumor regression at day 27, respectively.

These results demonstrate that the combination therapy resulted in greater amount of tumor growth inhibition compared to either single agent alone demonstrating enhanced in vivo anti-tumor efficacy of the combination against EGFR exon19del (E746_A750del) mutation expressing cancer.

1. A method of treating cancer in a subject in need thereof, comprising administering to the subject a therapeutically effective amount of a combination of the SOS1 inhibitor: 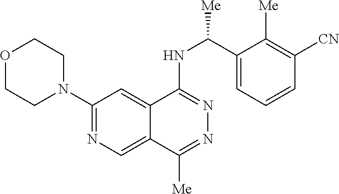 [SMILES: Cc1c(C#N)cccc1[C@@H](C)Nc1nnc(C)c2cnc(N3CCOCC3)cc12]
(R)-2-methyl-3-(1-((4-methyl-7-morpholinopyrido[3,4-d]pyridazin-1-yl)amino)ethyl)benzonitrile or a pharmaceutically acceptable salt thereof, and an EGFR inhibitor.

2. The method according to claim 1, wherein the EGFR inhibitor is selected from osimertinib, gefitinib, erlotinib, afatinib, brigatinib, icotinib, cetuximab, or a pharmaceutically acceptable salt thereof.

3. The method according to claim 1, wherein the EGFR inhibitor is osimertinib.

4. The method according to claim 1, wherein the EGFR inhibitor is gefitinib.

5. The method according to claim 1, wherein the EGFR inhibitor is erlotinib.

6. The method according to claim 1, wherein the EGFR inhibitor is cetuximab.

7. The method according to claim 1, wherein the SOS1 inhibitor and the EGFR inhibitor are administered on the same day.

8. The method according to claim 1, wherein the SOS1 inhibitor and the EGFR inhibitor are administered on different days.

9. The method according to claim 1, wherein the SOS1 inhibitor is administered at a maximum tolerated dose.

10. The method according to claim 1, wherein the EGFR inhibitor is administered at a maximum tolerated dose.

11. The method according to claim 1, wherein the SOS1 inhibitor and the EGFR inhibitor are each administered at a maximum tolerated dose.

12. The method according to claim 1, wherein the EGFR inhibitor is administered at below maximum tolerated dose.

13. The method according to claim 1, wherein the SOS 1 inhibitor is administered at below maximum tolerated dose.

14. The method according to claim 1, wherein the SOS1 inhibitor and the EGFR inhibitor are each administered at below maximum tolerated dose.

15. The method according to claim 1, wherein the therapeutically effective amount of the combination of the SOS1 inhibitor and the EGFR inhibitor results in an increased duration of overall survival, an increased duration of progression free survival, an increase in tumor growth regression, an increase in tumor growth inhibition or an increased duration of stable disease in the subjects relative to treatment with only the SOS1 inhibitor.

16. The method according to claim 1, wherein the therapeutically effective amount of the combination of the SOS1 inhibitor and the EGFR inhibitor results in an increased duration of overall survival, an increased duration of progression free survival, an increase in tumor growth regression, an increase in tumor growth inhibition or an increased duration of stable disease in the subjects relative to treatment with only the EGFR inhibitor.

17. A pharmaceutical composition, comprising a therapeutically effective amount of a combination of a SOS1 inhibitor and an EGFR inhibitor according to claim 1, and a pharmaceutically acceptable excipient.

18. A method for inhibiting SOS1 activity in a cell, comprising contacting the cell in which inhibition of SOS1 activity is desired with an effective amount of a combination the SOS1 inhibitor: 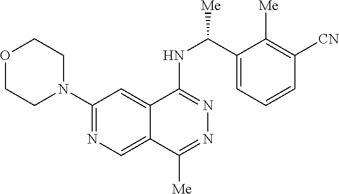 [SMILES: Cc1c(C#N)cccc1[C@@H](C)Nc1nnc(C)c2cnc(N3CCOCC3)cc12]
(R)-2-methyl-3-(1-((4-methyl-7-morpholinopyrido[3,4-d]pyridazin-1-yl)amino)ethyl)benzonitrile or a pharmaceutically acceptable salt thereof, and an EGFR inhibitor.

19. The method according to claim 18, wherein the EGFR inhibitor is selected from osimertinib, gefitinib, erlotinib, afatinib, brigatinib, icotinib, cetuximab, or a pharmaceutically acceptable salt thereof.

20. The method according to claim 18, wherein the EGFR inhibitor is osimertinib.

21. The method of according to claim 18, wherein the EGFR inhibitor is gefitinib.

22. The method according to claim 18, wherein the EGFR inhibitor is erlotinib.

23. The method according to claim 18, wherein the EGFR inhibitor is cetuximab.

24. The method according to 1, wherein the EGFR inhibitor synergistically increases the sensitivity of cancer cells to the SOS1 inhibitor.

25. A method for increasing the sensitivity of a cancer cell to a SOS1 inhibitor comprising administering to a subject undergoing treatment with an effective amount of a combination the SOS1 inhibitor: 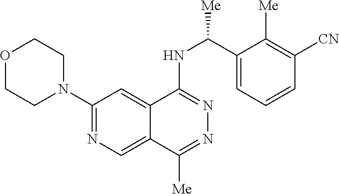 [SMILES: Cc1c(C#N)cccc1[C@@H](C)Nc1nnc(C)c2cnc(N3CCOCC3)cc12]
or a pharmaceutically acceptable salt thereof, and an EGFR inhibitor, wherein the EGFR inhibitor synergistically increases the sensitivity of the cancer cell to the SOS1 inhibitor.

26. The method according to claim 1, wherein the therapeutically effective amount of the SOS1 inhibitor in the combination is between about 0.01 to 100 mg/kg per day.

27. The method of claim 26, wherein the therapeutically effective amount of the SOS1 inhibitor in the combination is between about 0.1 to 50 mg/kg per day.

28. The method according to claim 1, wherein the therapeutically effective amount of the EGFR inhibitor in the combination is between about 0.01 to 100 mg/kg per day.

29. The method of claim 28, wherein the therapeutically effective amount of the EGFR inhibitor in the combination is between about 0.1 to 50 mg/kg per day.

30. The method according to claim 1, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

31. The method of claim 30, wherein the cancer wherein the cancer is a SOS1-associated cancer.

32. The method of claim 30, wherein the cancer is a KRas G12C-associated cancer.

33. The method of claim 30, wherein the cancer is selected from the group consisting of lung cancer, leukemia, pancreatic cancer, colorectal cancer and uterine cancer.

34. The method of claim 33, wherein the lung cancer is lung adenocarcinoma.

35. The method of claim 33, wherein the lung cancer is non-small cell lung cancer.

36. The method of claim 33, wherein the leukemia is cute myeloid leukemia (AML).

37. A kit comprising the pharmaceutical composition of claim 17 for treating SOS1-associated cancer in a subject.

38. The kit according to claim 37, further comprising an insert with instructions for administration of the pharmaceutical composition(s).
